(19) United States
(12) Patent Application Publication
(10) Pub. No.: US 2020/0214579 A1
(43) Pub. Date: Jul. 9, 2020

(54) SENSOR AND METHODS FOR CONTINUOUS NON-INVASIVE BLOOD PRESSURE MEASUREMENT AND CARDIOVASCULAR HEMODYNAMICS MONITORING IN HEALTHCARE, REHABILITATION AND WEARABLE WELLNESS MONITORS

(71) Applicant: THE BOARD OF TRUSTEES OF THE UNIVERSITY OF ILLINOIS, Urbana, IL (US)

(72) Inventors: Shane Phillips, Wheaton, IL (US); Mohamed Ali, Chicago, IL (US)

(21) Appl. No.: 16/825,673

(22) Filed: Mar. 20, 2020

Related U.S. Application Data
(63) Continuation-in-part of application No. PCT/US18/52144, filed on Sep. 21, 2018.
(60) Provisional application No. 62/561,802, filed on Sep. 22, 2017.

(51) Int. Cl. A61B 5/021; A61B 5/0245; A61B 5/00

(52) U.S. Cl. CPC A61B 5/02141; A61B 5/0245; A61B 5/02125; A61B 5/6831; A61B 5/6832; A61B 5/6804

(57) ABSTRACT

An example embodiment includes a blood pressure monitor system configured to continuously monitor blood pressure. The blood pressure monitor system includes a housing, a sensor arranged in a first side of the housing, at least one light emitting diode arranged in the first side of the housing, a barrier coupled to the housing and arranged between the sensor and the at least one light emitting diode, wherein the barrier is opaque, and a processor in communication with the sensor, the processor configured to continuously determine a blood pressure based on a reflected light.

### CROSS-REFERENCE TO RELATED APPLICATIONS

This application claims priority to PCT application serial no. PCT/US18/52144, filed Sep. 21, 2018, which claims priority to U.S. Provisional Pat. App. No. 62/561,802, filed Sep. 22, 2017, titled “Sensor for Continuous Non-Invasive Blood Pressure Measurement and Cardiovascular Monitoring,” all of which are incorporated herein by reference in their entireties.

### FIELD OF THE DISCLOSURE

The disclosure is related to continuous noninvasive cardiovascular hemodynamics monitoring devices, and in particular to continuous noninvasive blood pressure monitors that utilize a multispectral photo pulse plethysmography sensor.

### BACKGROUND

Wearable continuous cardiovascular and physical activity monitors are becoming an integral part of comprehensive health management programs aimed at reducing hospital readmission, improving adherence to home- and tele-rehabilitation programs and increasing patients' compliance and engagement in their wellness management plans. These devices will increase cost effectiveness of tele-rehabilitation, and promote reimbursable clinical services. The most sought after features of this class of devices include unobtrusiveness, accuracy, ease of use, patient comfort, and medical cost reduction.

Continuous monitoring of cardiovascular parameters including continuous blood pressure and continuous cardiac output is an essential part of intensive care and anesthesia monitoring, an integral component of sleep studies, ambulatory blood pressure monitoring, and recently continuous cardiovascular monitoring is increasingly required during cardiovascular rehabilitation and post discharge health management programs. Yet, the gold standard of continuous cardiovascular monitoring is invasive recording of blood pressure and hemodynamic parameters.

Previously, attempts at non-invasive continuous cardiovascular monitors have not met the accuracy and repeatability (precision) requirements to be used in critical healthcare applications due to the lack of their ability to measure the essential hemodynamic parameter of Vascular Resistance. The use of such devices for wearable continuous hemodynamic monitors are hindered by their bulky size, obtrusiveness, high total cost of ownership, and other factors related to their accuracy and motion tolerance.

There are three technologies that predominate continuous monitoring of cardiovascular hemodynamic parameters. These include Pulse Transit Time (“PTT”) based algorithms, applanation tonometry and algorithms that use transfer function to reconstruct systemic pressure wave from volume clamped digital pulse wave. The PTT algorithms predict systemic pressure from the time that the recorded infrared photoplethysmography pulse wave takes to arrive at the peripheral recording site measured from the R wave of the ECG. Although a high correlation exists between this parameter and Systolic Pressure, there are problems with accuracy. This method has limitations due to the deterioration of accuracy and repeatability especially in the case of co-existing cardiovascular disease conditions or under the use of vasoactive drugs. This method is very sensitive to motion artifacts. The method is also known to be associated with very high drift in the calculated pressure and requires very frequent calibration with ECG and blood pressure cuff readings. Also, it cannot be used without ECG since R wave syncing is the only way to measure Pulse Transit Time.

The second method is based on applanation tonometry in which a probe that consists of a piezoelectric element is placed on an artery, usually a radial artery at the wrist or a carotid artery at the neck, to record the amplitude (or the force) of the pulsewave. Processors calculate systemic pressure from the recorded pulsewave using frequency-domain transfer functions. This method is very sensitive to the placement of the probe, intolerant to motion and very dependent on the operator (requires physical placement of the probe during data acquisition). This method is reserved for research applications and very limited clinical applications in which a trained operator is available to record the pulse wave.

The third method is based on volume clamp using finger probes consisting of an infrared sensor and pressure cuffs in which the inflation pressure is controlled through a fast feedback control loop to maintain a fixed pulse wave volume in the probed fingers. A processor uses the clamp pressure to calculate the central pressure and stroke volume. Devices based on this method have limitations that limit their use by the public and in medical care. The most important is related to precision and repeatability. These technologies are bulky and the finger probes cause discomfort to the patients due to the pressure from volume clamp cuffs that are used. Errors in measurement due to motion with exercise are common and similar to the other technologies. There is also no adaptation for the change in patient orientation.

All continuous blood pressure methods are calibrated for recumbent patients in supine positions. This limits the usability of these methods in rehabilitation and exercise medicine where continuous monitoring is required during physical activity and repeated changes in patient position. The major pitfall of all noninvasive technologies is the unmeasured change in the vascular tone of resistance arterioles and capillaries and the venular capacitance. The vascular autoregulatory mechanism is controlled by the sympathetic tone and is further influenced by vasoactive drugs and circulating hormones.

### SUMMARY OF THE INVENTION

In a first aspect, the present disclosure provides a blood pressure monitoring system that includes: (a) a housing; (b) a sensor arranged in the housing; (c) at least one light source arranged in the housing; and (d) a processor in communication with the sensor, the processor configured to continuously determine a blood pressure based on a reflected light from light emitted by the at least one light source and received by the sensor.

In a second aspect, the present disclosure provides a method for continuously monitoring blood pressure that includes: (a) securing a blood pressure monitor system according to any one of claims 1-20 to an external surface of a body; (b) emitting, via the at least one light source, light towards the external surface of the body; (c) receiving, via the sensor, a reflected light from the body; (d) sending to the processor, via the sensor, information related to the reflected light; (e) receiving, via the processor, the information related to the reflected light; and (f) continuously determining, via the processor, a blood pressure based on the information related to the reflected light.

In a third aspect, the present disclosure provides a tangible, non-transitory computer-readable medium having instructions encoded thereon, wherein the instructions, when executed by the processor, cause a blood pressure monitor system according to the first aspect to perform a method comprising: (a) emitting, via the at least one light source, light towards the external surface of the body; (b) receiving, via the sensor, a reflected light from the body; (c) sending to the processor, via the sensor, information related to the reflected light; (d) receiving, via the processor, the information related to the reflected light; and (e) continuously determining, via the processor, a blood pressure based on the information related to the reflected light.

These as well as other aspects, advantages, and alternatives, will become apparent to those of ordinary skill in the art by reading the following detailed description, with reference where appropriate to the accompanying drawings.

### BRIEF DESCRIPTION OF THE DRAWINGS

Features, aspects, and advantages of the presently disclosed technology may be better understood with regard to the following description, appended claims, and accompanying drawings where:

FIG. 1 shows a blood pressure monitor system, according to an example embodiment;

FIG. 2 shows a blood pressure monitor system, according to an example embodiment;

FIG. 3 shows a blood pressure monitor system, according to an example embodiment;

FIG. 4 shows a three-lobe blood pressure monitor system, according to an example embodiment;

FIG. 5 shows a plot obtained from the measurement of reflected light from the blood pressure monitor system, according to an example embodiment;

FIG. 6 shows an example Dicrotic notch, according to an example embodiment;

FIG. 7 shows a normal arterial blood pressure waveform and its relation to the electrocardiographic R wave, according to an example embodiment;

FIG. 8 shows a graphical user interface displaying output of the blood pressure monitoring system, according to an example embodiment;

FIG. 9 shows an example method according to some embodiments; and

FIG. 10 shows a blood pressure monitor system, according to an example embodiment.

The drawings are for the purpose of illustrating examples, but it is understood that the inventions are not limited to the arrangements and instrumentalities shown in the drawings.

### DETAILED DESCRIPTION

The foregoing and other features and advantages of the invention are apparent from the following detailed description of exemplary embodiments, read in conjunction with the accompanying drawings. The detailed description and drawings are merely illustrative of the invention rather than limiting, the scope of the invention being defined by the appended claims and equivalents thereof.

Embodiments of the invention will now be described with reference to the Figures, wherein like numerals reflect like elements throughout. The terminology used in the description presented herein is not intended to be interpreted in any limited or restrictive way, simply because it is being utilized in conjunction with detailed description of certain specific embodiments of the invention. Furthermore, embodiments of the invention may include several novel features, no single one of which is solely responsible for its desirable attributes or which is essential to practicing the invention described herein.

The use of the terms “a” and “an” and “the” and similar referents in the context of describing the invention are to be construed to cover both the singular and the plural, unless otherwise indicated herein or clearly contradicted by context. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof.

Recitation of ranges of values herein are merely intended to serve as a shorthand method of referring individually to each separate value falling within the range, unless otherwise indicated herein, and each separate value is incorporated into the specification as if it were individually recited herein. The word “about,” when accompanying a numerical value, is to be construed as indicating a deviation of up to and inclusive of 10% from the stated numerical value. The use of any and all examples, or exemplary language (“e.g.” or “such as”) provided herein, is intended merely to better illuminate the invention and does not pose a limitation on the scope of the invention unless otherwise claimed. No language in the specification should be construed as indicating any nonclaimed element as essential to the practice of the invention.

References to “one embodiment,” “an embodiment,” “example embodiment,” “various embodiments,” etc., may indicate that the embodiment(s) of the invention so described may include a particular feature, structure, or characteristic, but not every embodiment necessarily includes the particular feature, structure, or characteristic. Further, repeated use of the phrase “in one embodiment,” or “in an exemplary embodiment,” do not necessarily refer to the same embodiment, although they may.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

### I. Overview

This disclosure is directed to exemplary systems including sensors and methods for the estimation of continuous noninvasive blood pressure through an approach that overcomes the limitations of currently available physiologic monitors at predicting continuous noninvasive blood pressure during physical activity and common activities of daily living. The exemplary systems and methods are practical for continuous home physiological monitoring and cardiac rehabilitation applications. The systems and methods advantageously increase the accuracy of blood pressure measures, are convenient ambulatory use, and enhance the validity of blood pressure and heart function measures during physical activity.

This disclosure uses photoplethysmography that utilizes the principal of differential light absorbance by different tissues and blood components to measure the volume change that occurs due to the pulse wave on each cardiac cycle. To measure the volume change, a probe is used that includes at least one light emitter and a light sensor to measure the absorbance of light versus time and the plethysmograph wave is extracted.

This disclosure describes a highly accurate sensor assembly used with multispectral pulse plethysmography (“msPPG”) to compute continuous blood pressure and other cardiovascular hemodynamic parameters.

To overcome the limitations of currently existing technologies of continuous noninvasive cardiovascular monitors, the physiology of the microcirculation and the role of the microcirculation in regulating blood pressure was leveraged. The systems and methods described herein take into account the vascular physiology and hemodynamics of the microcirculation which is the major contributor to systemic vascular resistance and eventually to the systemic blood pressure.

This system described herein comprises a multispectral photo Pulse plethysmography (“msPPG”) sensor assembly that records the pulse waveform at multiple wavelengths to record and measure the phase or temporal shifts between pulse waves recorded at different wavelengths as blood flows from deeper arteries and arterioles to superficial capillaries, thus measuring and registering the local pulse wave propagation time in the microcirculation as measured by multispectral plethysmography. Also the difference in intensity, flux, volume, quantity, or magnitude of the pulsewave at different wavelengths is measured and recorded for analysis.

In an example embodiment, a blood pressure monitor system includes a housing, a sensor arranged in the housing, at least one light emitting diode arranged in the housing, and a processor in communication with the sensor, the processor configured to continuously determine a blood pressure based on a reflected light from light emitted by the at least one light source and received by the sensor. The barrier is opaque.

In another example embodiment, a method of using the blood pressure monitor system includes securing the blood pressure monitor system to an external surface of a body. The method further includes emitting, via the at least one light source, light towards the external surface of the body. The method further includes receiving, via the sensor, a reflected light from the body. The method further includes sending to the processor, via the sensor, information related to the reflected light. The method further includes receiving, via the processor, the information related to the reflected light. The method further includes continuously determining, via the processor, a blood pressure based on the information related to the reflected light.

### II. Example Blood Pressure Monitoring Device

The exemplary systems and methods described above may be implemented via a blood pressure monitoring system configured to accurately and continuously detect the blood pressure of a patient. In an example embodiment, a blood pressure monitor system includes a housing, a sensor arranged in the housing, at least one light source arranged in the housing, and a processor in communication with the sensor, the processor configured to continuously determine a blood pressure based on a reflected light from light emitted by the at least one light source and received by the sensor.

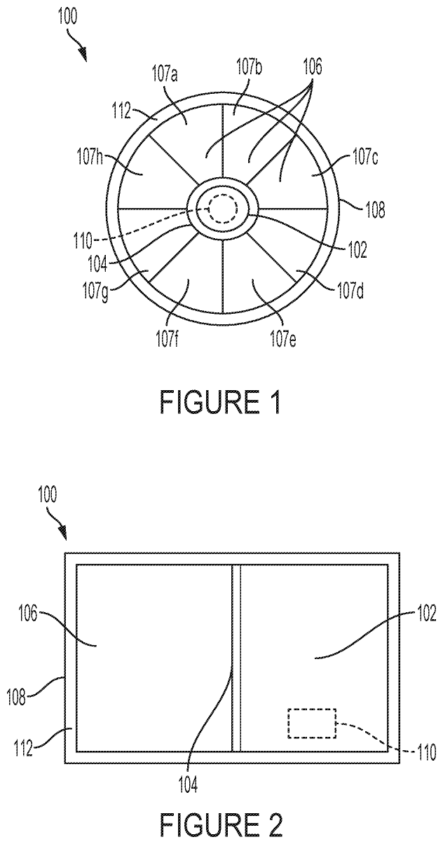

FIG. 1 shows an example configuration of a blood pressure monitor system 100. Blood pressure monitor system 100 includes a housing 108, a sensor 102 arranged in the housing 108, at least one light source 106 arranged in the housing 108 and a processor 110 in communication with the sensor 102. The processor 110 is configured to continuously determine a blood pressure based on a reflected light from light emitted by the at least one light source 106 and received by the sensor 102.

In one embodiment, the housing 108 may be opaque to prevent light external to the blood pressure monitor 100 from interfering with the sensor 102. In addition, the housing 108 may be made of a material that is waterproof and resilient. The housing 108 and the sensor 102 may be coated in clear epoxy or clear silicone, for example, to seal and protect the housing 108 and the sensor 102 from fluids such as sweat or water. This provides the benefit of permitting the use when a patient is exercising, for example. In one embodiment, the housing 102 may include slots to receive additional hardware configured to communicate with the processor 110 to increase functionality of the blood pressure monitor 100.

In one embodiment, sensor 102 may be a multispectral sensor configured to receive and collect reflected light on multiple spectrums. In other words, the multispectral sensor may receive and collect many wavelengths of light including, but not limited to, red, green and blue wavelengths of light, as well as infrared radiation and ultraviolet light (non-visible wavelengths). Sensor 102 may also include a multispectral array of photodiode sensors.

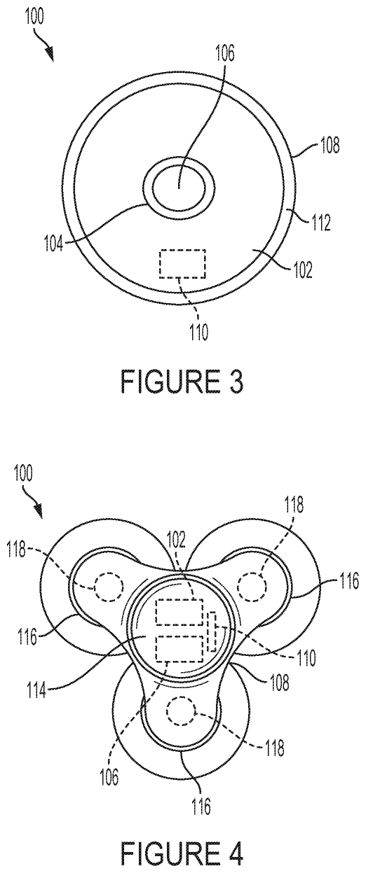

The at least one light source 106 is configured to emit or shine a light on an external portion of the body of a patient. In one embodiment, the at least one light source 106 is a single light emitting diode (“LED”) that is a broad-spectrum white LED 106, shown in FIGS. 2 and 3. In some embodiments, shown in FIG. 1, the at least one light source 106 includes a plurality of LEDs 107a-h each corresponding to a single color of a light spectrum (e.g., violet, blue, green, yellow, orange, red and infrared) such that the plurality of LEDs each corresponds to a different wavelength of light. In these embodiments, the different wavelength of light for each of the plurality of LEDs ranges from 425 nm to 1100 nm on the light spectrum. Any combination of two or more light wavelengths in the foregoing range that are adequately spaced apart may be utilized to determine blood pressure. Example functional combinations of light pulses having wavelengths include, but are not limited to, (i) 435 nm, 550 nm and 690 nm, (ii) 460 nm, 570 nm and 710 nm, or (iii) 515 nm, 710 nm and 950 nm.

In one embodiment, the sensor 102 is arranged in a first side 112 of the housing 108 and the at least one light source 106 is arranged adjacent to the sensor 102 on the first side 112 of the housing 108. In this embodiment, the housing 108 has a barrier 104 arranged between the sensor 102 and the at least one light source 106. The barrier 104 is opaque thereby preventing light emitted from the at least one light source 106 from being received via sensor 102 prior to the emitted light being reflected from a body. In one embodiment, the barrier 104 may be part of the housing 108. Alternatively, the barrier 104 may be made of a different material than the housing and may encase one or both of the sensor 102 and the light source 106. In one embodiment, sensor 102 and light source 106 are spaced apart from each other a distance ranging from 2 mm to 5 mm. The separation provided by the barrier 104 may beneficially reduce noise or interference at the sensor 102, thereby increasing accuracy in measurement of the reflected light.

As shown in FIG. 1, sensor 102, barrier 104, and light source 106 can be configured in a circular housing 108. The first side 112 of the housing 108 is substantially flat or may be curved to adapt the blood pressure monitor 100 to a patient's body. Light source 106 is arranged surrounding sensor 102 in a first side of the housing. In this embodiment, the light source 106 is a plurality of LEDs 107a-h, described above, and the plurality of LEDs 107a-h are arranged in a ring about a perimeter of the sensor 102. The barrier 104 is ring shaped and disposed in between sensor 102 and light source 106. This configuration may beneficially allow the barrier 104 to efficiently prevent the emitted light from light emitting diode 106 from being received via sensor 102 prematurely. In some embodiments, the sensor 102 is arranged surrounding light source 106, with barrier 104 disposed in between. In various other embodiments, the sensor 102 and the light source 106 may have a combined footprint smaller than the first side 112 of the housing 108.

FIG. 2 shows another example configuration of a blood pressure monitor 100. In FIG. 2, the sensor 102 and the light source 106 are arranged side by side in the first side 112 of the housing 108. The barrier 104 is disposed in between the sensor 102 and the light source 106 in the first side 112 of the housing 108.

FIG. 3 shows another example configuration of blood pressure monitor 100. In FIG. 3, sensor 102 is arranged surrounding the light source 106 in the first side 112 of the housing 108. The sensor 102 takes the form of a ring and the barrier 104 is ring-shaped and disposed in between the sensor 102 and the light source 106. In this configuration, the light source 106 is a light emitting diode 106 that is a broad-spectrum white LED.

FIG. 4 shows another example configuration of blood pressure monitor 100. In FIG. 4, the blood pressure monitor system 100 includes a housing 108 with a central segment 114 from which three lobes 116 extend. The first surface (not shown) of housing 108 may be curved to adapt the blood pressure monitor system 100 to a surface of a patient's body. Alternatively, the housing 108 may be made of a material that permits flexion to adapt to a patient's body while still maintaining the functional arrangement of the sensor 102 and the light source 106. For example, any curvature in the first side of the housing 108 may be configured to adapt to the curvature of a patient's chest. Placement of the blood pressure monitor system 100 in this location on a patient's body torso has the advantage of measuring central blood pressure and stroke volume, rather than measurements recorded from a limb that may be impacted by the position of a patient's limb. The sensor 102 and the light source 106 are disposed within the central segment 114 of housing 108. The housing 108 may contain an adhesive disposed on each lobe 116 of the housing 108 that is configured to adhere the housing 108 to a surface of the patient's body. Each of the three lobes 116 are coupled to an ECG electrode 118 configured to record at least one channel of ECG. The three-lobed embodiment provides a spaced arrangement between the ECG electrodes 118 to avoid interference between the ECG electrodes 118. In one embodiment, the ECG electrodes 118 may be removably coupled to the housing with a snap-fit connector. The ECG electrodes 118 may be coupled to additional on-board sensors to measure other factors, such as heart rate, heart rate variability, energy expenditure, respiration, anaerobic and aerobic phase and power reserve running distance, vertical jump height, oxygen saturation (S_{O2}), continuous hemodynamics (i.e., blood pressure, stroke volume, AoPWV, cardiac load).

In one embodiment, the blood pressure monitor system 100 may include one or more of a transmitter, a receiver and a transceiver. As used herein, transceivers are devices that can both transmit and receive wireless communications, such as a combined radio transmitter and receiver that share common circuitry or a common housing. In an optional example in which the blood pressure monitor system 100 and a remote computing device (e.g., a computer, tablet, smart phone, smart watch, etc.) each have a transceiver, the transceiver of the blood pressure monitor system 100 and the transceiver of the remote computing device are capable of bi-directional wireless communication with each other. In an alternate example, both the transmitter and the receiver configured to send and receive wireless communications, respectively, have no common circuitry and may be used in place of the transceiver. Data may be transmitted from the blood pressure monitor system 100 to the remote computing device for further data processing or remote data storage. In addition, the remote computing device may transmit instructions or data based on patient-specific information, for example, to adjust thresholds or other parameters utilized by processor 110.

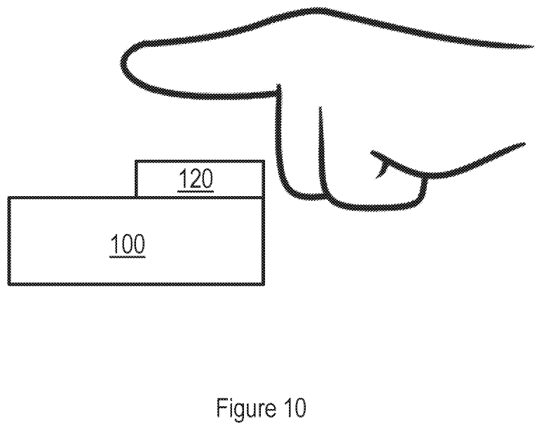

The combination of the multispectral plethysmography acquisition with onboard accelerometer data to report body orientation and the ECG to calculate Aortic pulse wave velocity (“AoPWV”) in addition to body temperature may increase accurate information about blood flow in the cardiovascular system that can be used by the processor 110 to predict or estimate the hemodynamic parameters. In addition, as shown in FIG. 10, inclusion of a Force Sensitive Resistor 120 or a load sensing component with the sensor 102 or the housing 108 will allow measurement of the contact pressure to the skin as measured by a Force Sensitive Resistor (“FSR”) 120 to correct and account for the contact pressure between the sensor 102 and the skin or body tissue.

Combining this technique with pulse wave velocity and pulse wave form analysis may increase the accuracy of estimated central pressure (i.e., Aortic pressure), Stroke Volume, Cardiac Load, Mean Arterial Pressure (“MAP”), and Total Peripheral Resistance. This modification can be added to any reflective or transillumination plethysmography sensor to provide feedback on the contact pressure of the sensor 102 against the skin or body part. For example, this modification can be integrated into wrist bands, oximeter finger probes, chest patches, arm bands, chest bands or any other optical sensor that measures a physiological, vital, or biochemical parameter from the body through contact with the skin or any other body tissue or organ.

In one embodiment, the blood pressure monitor system 100 may be coupled to one or more bands to retain the housing 108 in place on a body. For example, the ends of a first elastic band may be coupled to opposite sides of the housing 102 and the band may wrap around a patient's chest to hold the housing 102 against the body. In another embodiment, the blood pressure monitor system 100 may be integrated in a form-fitting garment such that the housing 108 is coupled to the garment and retains the housing 108 in place on a body such that the sensor 102 and the light source 106 are unobstructed relative to the body.

In one embodiment, the sensor 102 is a high speed multispectral camera or RGB camera (i.e., color camera) and the light source 106 is a broadband white LED to be used for illumination. The RGB camera acts as a two dimensional array of photosensors that may be used to calculate the time of propagation as well as relative and temporal differences in pulsewave folume, flux, intensity, or magnitude of the pulsewave cycle from deeper arterioles to the shallower capillaries and to estimate blood pressure based on this calculation.

In various other embodiments, the blood pressure monitor system 100 may further include an array of trans-impedance amplifiers. The array of trans-impedance amplifiers may have a low noise and high common mode rejection ratio. The array of trans-impedance amplifiers may amplify the signal from the photodiode arrays. In addition, or alternatively, the blood pressure monitor 100 may further include at least one analog to digital converter configured to digitize recorded signals for each pulse wave of light emitted from the at least one light source at a sampling rate of at least 250 samples per second for each of the recorded pulse waves of light. In still further embodiments, the blood pressure monitor system 100 may also include an accelerometer coupled to the housing 108 and in communication with the processor 110. The accelerometer may be used to calibrate the blood pressure monitor system 100 or provide feedback information about the body orientation to the processor 110 that may be used in the hemodynamic parameters estimation. In yet another embodiment, the blood pressure monitor system 100 may include a power source that may be wirelessly re-charged.

In another embodiment, blood pressure monitor system 100 may further include a second sensor arranged opposite to the at least one light source 106 such that a portion of a body may be disposed between the at least one light source 106 and the second sensor. For example, the first sensor 102 may be arranged with the light source 106 on one side of a finger to measure reflected light and the second sensor may be arranged on the opposite side of the finger to measure transillumination light.

### III. Example Operating Environment

In healthy elastic arteries, the reflected pulse wave (of blood) from peripheral circulation is slower than in the case of arteries with stiffness, thus the reflected pulse wave does not augment the pressure wave as the reflection wave meets the forward wave towards its end. However, in less elastic vessels with increased stiffness attributed to matrix remodeling or atherosclerosis, the reflected pulse wave is faster and meets the forward wave during its buildup or rising phase, thus the reflected wave augments the forward wave and raises the pulse pressure.

The higher amplitude forward waveform and the lower amplitude reflection waves can be extracted by decomposing the PPG waveform into its fundamental waves (or harmonics). This may be achieved using Digital Signal Processing Techniques including Wavelet Transform, FFT/Inverse FFT, Eigenvectors, Eigenvalues, Principle Component analysis, Independent Component Analysis, and other techniques.

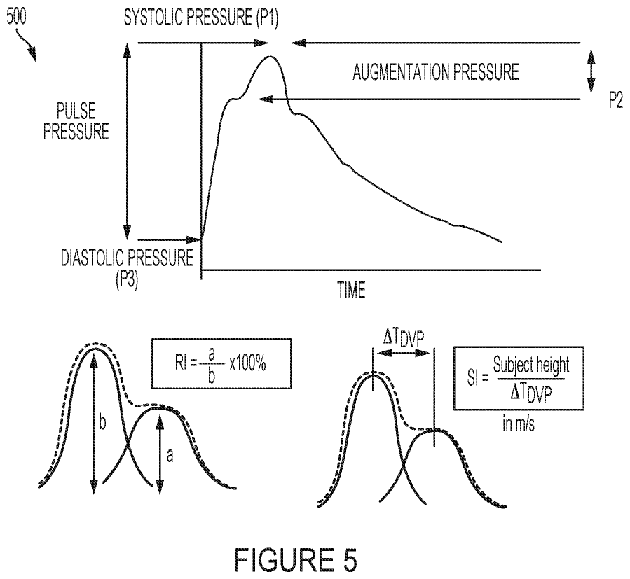

FIG. 5 shows a plot obtained from the measurement of reflected light from the blood pressure monitor 100, according to an example embodiment. The measurement may yield a multitude of parameters, including the Augmentation Index, the Reflection Index, and the Stiffness Index. The Augmentation index is a measure of systemic arterial stiffness derived from the ascending aortic pressure waveform. The Reflection Index (“RI”), shown in FIG. 5, indicates the relative ratio between forward pulse wave amplitude and the reflected wave amplitude.

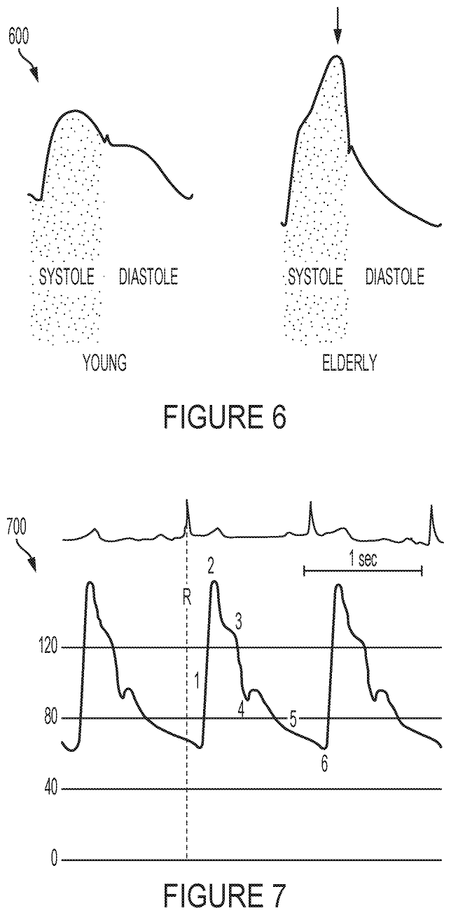

FIG. 6 shows an example dicrotic notch, according to an example embodiment. The dicrotic notch marks the event of aortic valve closure which causes a small brief change in pressure as a result of the movement of aortic valve leaflets in the opposite direction to the pulse wave flow vector. The timing, depth and duration of the dicrotic notch are related to the systolic blood pressure that affects the timing and the velocity of aortic valve closure. The dicrotic notch on the PPG demarcates the boundary between the systolic phase and the diastolic phase of the pulse wave. The contour and the slope of each phase together with the time integral of each phase (or area under the curve) are affected by the elasticity of the blood vessels and the internal pressure. These parameters can be used in conjunction with the local pulse wave propagation time as well as differences in pulsewave flux, volume, intensity, quantity, or magnitude in the microcirculation as measured by multispectral plethysmography to accurately estimate hemodynamic parameters including blood pressure and stroke volume.

FIG. 7 shows a normal arterial blood pressure waveform and its relation to the electrocardiographic R wave, according to an example embodiment. In FIG. 7, the systolic upstroke (1), systolic peak pressure (2), systolic decline (3), dicrotic notch (4), diastolic runoff (5), and end-diastolic pressure (6) are all shown.

In some embodiments, sensor 102 from FIGS. 1-4 may be a specialized multispectral photo Pulse plethysmography (“msPPG”) sensor assembly that records the pulse waveform at multiple wavelengths to study the phase or temporal shifts or propagation time between pulse waveform cycles recorded at different wavelengths of light as blood flows from deeper arteries and arterioles to superficial capillaries. Also the difference in volume, quantity, intensity, flux, or magnitude of the pulsewave at different wavelengths is measured and recorded for analysis.

As described above, the blood pressure monitor system 100 of FIGS. 1-4 includes a processor that may be configured to continuously calculate the change in pulse wave blood volume and the impedance to blood flow by measuring the change in time (phase or temporal) shifts between pulse waveforms recorded at different wavelengths as well as the difference in volume, quantity, flux, intensity, or magnitude of the pulsewave at different wavelengths

In some embodiments, signal processing techniques may be applied to the high-resolution pulse waveform cycle data recorded at multiple wavelengths at a high sampling rate. A high sampling rate may be more than 250 samples per second at adequate resolution not less than 12 bit effective resolution. A feature matrix may be constructed from the acquired multispectral waveforms. This may be passed on as input to a machine learning (“ML”) algorithm that predicts changes in blood pressure from the combined analysis of morphological features and time-frequency domain analysis of the multispectral pulse waveforms. During a continuous noninvasive blood pressure (“cNIBP”) prediction and estimation step of algorithm workflow, the ML algorithm incorporates a model constructed from the cardiovascular hemodynamic data recorded from human subjects. The sensor 102 and associated processor allow the simultaneous measurement of blood volume changes and the impedance (resistance+capacitance) to the blood flow from a single point of measurement on the human body, capturing a real-time blood pressure measurement.

Exemplary blood pressure monitor system 100 is capable of obtaining a high resolution and high sampling rate recording of a multispectral plethysmogram. This is achieved by shining a white light LED 106 of suitable spectral composition onto the recording site (e.g., a portion of a patient's body). A multispectral sensor 102 is used to resolve the white light reflected by the illuminated site into multiple wavelengths that spans the red, green, and blue parts of the light spectrum. The acquired pulse waveform cycles are analyzed for their timing and phase shifts that represent different arrival times of blood pulse wave at different depths of the illuminated tissue, and the difference in volume, quantity, flux, intensity, or magnitude of the pulsewave at different wavelengths. This is due to the fact that different wavelengths of light penetrate through different depths of illuminated tissues.

For example, the blue spectrum of light does not penetrate through deep tissue and reflects from the surface of the skin, while the red spectrum can penetrate a few millimeters deeper into the illuminated tissue and is capable of arriving at and reflecting from deeper arteries and arterioles. The time that the pulse wave cycle takes to propagate from deeper arterioles to the shallower capillaries can be measured by measuring the time differences between pulse waveform cycles recorded at the red spectrum to those recorded at the blue spectrum, respectively. Infrared light can even penetrate deeper into the tissues and can be used to record pulse waveform cycle propagation starting from a deeper segment of the tissue. The time it takes for the pulse waveform cycle to propagate from deeper arteries and arterioles to shallower capillaries is directly correlated to the vascular tone and resistance, thus it is very promising as a measure of peripheral resistance that is a determinant of arterial blood pressure. Also, the measurement of temporal variation in volume, quantity, flux, intensity, or magnitude of the pulsewave at different wavelengths using the same principal allows the measurement of the relative resistance of smaller capillaries at shallower depth as registered in the blue or green spectrum versus larger arterioles and arteries as registered in the red and infrared spectrum which is related to the change of capillary tone over time and is a factor that affects blood pressure.

### IV. Example Measurements

A custom PPG sensor consisting of a broad spectrum high intensity white LED that was used to shine light through the proximal phalanx of the left hand middle finger or the thumb. This light source was developed by affixing a proprietary blend of up conversion “Luminophores” to the face of a high intensity blue LED to emit light with a broad spectral composition spanning the light spectral wavelengths of, for example, 425 nm to 1050 nm.

A multispectral array of photodiode sensors is used to measure the transillumination intensity and the reflected light intensity at ten different wavelengths; 460 nm, 490 nm, 515 nm, 560 nm, 615 nm, 660 nm, 700 nm, 805 nm, 950 nm and 1050 nm.

An array of trans-impedance amplifiers with low noise and high common mode rejection ratio was utilized to amplify the signal from the photodiode arrays.

In another embodiment, the multispectral PPG sensor was constructed of multiple light emitting diodes (“LEDs”) each emitting a single color of the light spectrum of different wavelengths within the spectral range of, for example, 450 nm to 1050 nm. In these embodiments, the individual light emitting diodes were pulsed (switched on, or activated) sequentially emitting the specific wavelength of light respective to the activated LED to illuminate or transilluminate a segment of the body tissue. The intensity of the reflected or transilluminated light is measured using at least one light sensor (or Photosensor) with a suitable spectral response and light sensitivity at the wavelength corresponding to the activated LED. The intensity of the reflected or transilluminated light emitted from each LED is measured and registered in the reflected light's respective wavelength.

The analog to digital conversion of the PPG waveforms was carried out using simultaneous sampling analog to digital converters of at least 16-bit resolution and at least 250 Hz (samples per second) sampling rate per channel to provide a high dynamic range and high temporal resolution conversion.

The PPG waveforms recorded at every wavelength were normalized, followed by averaging 10 to 15 PPG waveform cycles to remove respiration and motion artifacts and to increase signal to noise ratio (“SNR”). Parallel signal processing of the multispectral waveforms is started by calculating the first, second and third derivatives of the PPG waveforms. These derivatives are used to identify peaks, points of inflection and aberrancy (or symmetry of each point along the waveform).

The set of features extracted from the recorded pulse waveform cycles include timing of peaks and inflection points relative to the R-wave of a simultaneously recorded ECG. Also, the extracted features may include Volume Time Integral (“VTI” or area under the curve) of the systolic and diastolic segments of the PPG waveforms and total VTI, the analysis may also include calculating the slopes of the rising systolic limb and the slopes of the systolic decay and the diastolic runoff.

The recorded multispectral pulse plethysmogram exhibits temporal shifts or phase shifts between pulse plethysmograms measured at different wavelengths which indicates that the pulse wave photoplethysmogram recorded at different wavelengths could be influenced by different factors of the pulse wave. For example, one or more PPG waveforms might be more influenced by volume changes on the arterial/arteriolar side, while another group of wavelengths could be more influenced by changes on the venular/venous side. Nevertheless, one or more wavelengths could be reflecting intraluminal velocity and red blood cells' alignment more than others. Interstitial fluid changes could be detected better in the infrared recorded PPG waveforms as changes in the baseline or the Baseline component of the PPG.

Accordingly, an important feature of the multispectral PPG is the phase or timing shifts as well as pulsewave volume, intensity, or flux variation between waveforms recorded at different wavelengths, and these were calculated from the derivatives of the multispectral PPG relative to the ECG R-wave. Noteworthy is that the absolute value of temporal or phase shifts between pulse waves recorded at different wavelength is also affected by the heart rate and were corrected in the processor based on the heart rate to provide a heart rate corrected value or to include beat-to-beat interval in the blood pressure determination that take the phase or temporal shifts of the msPPG as an input.

Two external devices may be used to calibrate the sensor and provide orientation information. These are the oscillometric blood pressure module and the triaxial accelerometer module. The oscillometric blood pressure may be used to periodically provide a reference blood pressure measurement to correct any drift and to recalibrate the continuous blood pressure sensor. The triaxial accelerometer may be used to feedback the orientation of the body, which is an input parameter to the cNIBP determination to adjust for pressure changes due to changes in the orientation of the body.

The accelerometer may be used to provide quality information when the oscillometric blood pressure module will be used to measure blood pressure, in this way blood pressure measurements recorded at a high level of motion will be rejected and not used for calibration or correction.

The multispectral PPG sensor can be used in trans-illumination mode or reflective mode or a combination of both to detect blood flow patterns in superficial and deep tissues. Also, it can be used on different sites, for example the multispectral PPG sensor was successfully used to record multispectral PPG waveforms from the proximal phalanges of the middle finger and thumb, from interdigital creases, from the wrist in trans-reflective mode and from the anatomical snuff box in both trans-illumination and trans-reflective modes. Also the multispectral PPG sensor successfully recorded and measured multispectral plethysmograms from the torso and from the chest wall.

### V. Example Studies

A pilot study was conducted to record multispectral Plethysmograms msPPG and blood pressure data from healthy volunteers at rest and after exercise. The study protocol involved the acquisition of Multispectral Plethysmograms msPPG recorded from healthy subjects simultaneously with blood pressure data collected by a standard method including manual and automated oscillometric blood pressure measurements. There were twenty-six (26) subjects recruited with an average age of 39 (ranging from 24 to 49 years old).

Multispectral Pulse-Wave Plethysmograms (msPPG) capturing and logging for offline analysis was performed using a custom developed device and software that simultaneously records ECG, msPPG, and blood pressure using an oscillometric method. Manual blood pressure measurements were performed for consistency and comparison.

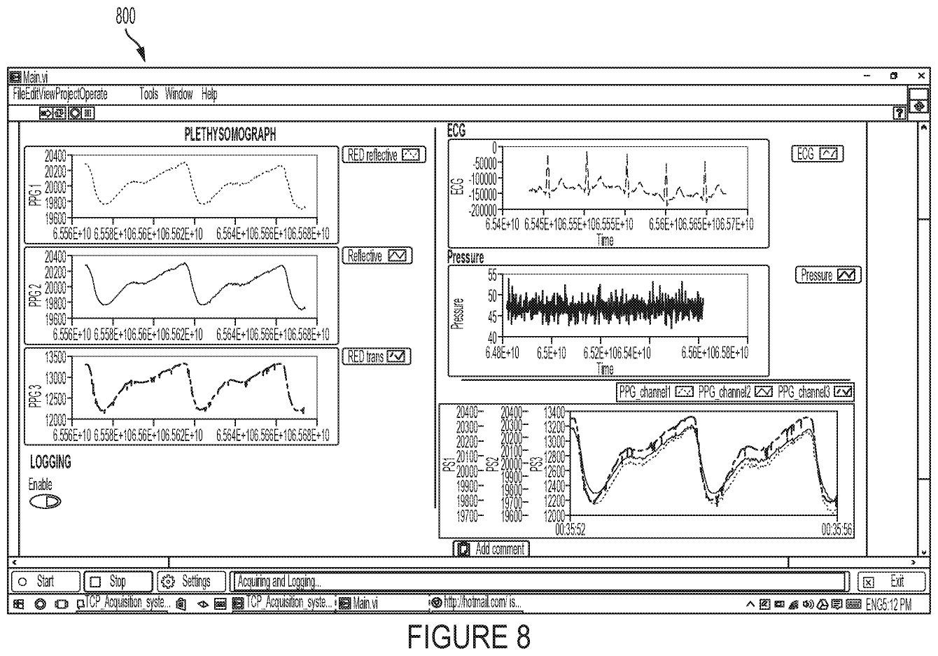

FIG. 8 shows a plot obtained from the measurement of reflected light from the blood pressure monitor, according to an example embodiment.

The research protocol includes the following: (1) continuous msPPG and blood pressure recording during aerobic exercise for signal quality assessment; (2) continuous data acquisition of msPPG, ECG, and blood pressure during recovery after aerobic exercise (treadmill); (3) stored data sets from each exercise session were analyzed using custom scripts running in MATLAB and/or Python to extract the time delays (i.e., phase or temporal shifts) between pulse waveform cycles recorded for different wavelengths as well as temporal variations in pulsewave volume, intensity, flux, or magnitude as described in the methods section above.

The resulting data points were plotted to determine the trend of the change in time shifts in msPPG versus blood pressure.

Results

Results from these studies include (1) a high correlation (r=0.97) between msPPG phase shift and systolic blood pressure; (2) a high correlation (r=0.85) between msPPG phase shift in diastolic runoff; (3) a high consistency and lack of drift between intrapersonal data between visits, which means that the calibration for each user is highly consistent and the same calibration will work over multiple days of use; (4) no drift in systolic blood pressure measurements versus msPPG phase shifts between different visits in the same person; (5) no drift in diastolic blood pressure measurements versus msPPG phase shifts between different visits in the same person; (6) a correlation between systolic blood pressure and msPPG time shifts for the same subject between different visits; (7) a correlation between diastolic blood pressure and msPPG time shifts for the same subject between different visits; and (8) a poor correlation between blood pressure measurements and msPPG shifts recorded from different individuals indicating the difficulty of finding a universal calibration method that can be used between different persons without the need for recalibration.

Of note, intrapersonal data points could be modeled through polynomial fitting, which means that calibration is straightforward and that heuristic methods, such as neural networks, or machine learning algorithms is not necessary.

Further, a poor correlation between interpersonal data and blood pressure data was determined, which means that the sensor and device will have to be calibrated for each user.

### IV. Example Methods

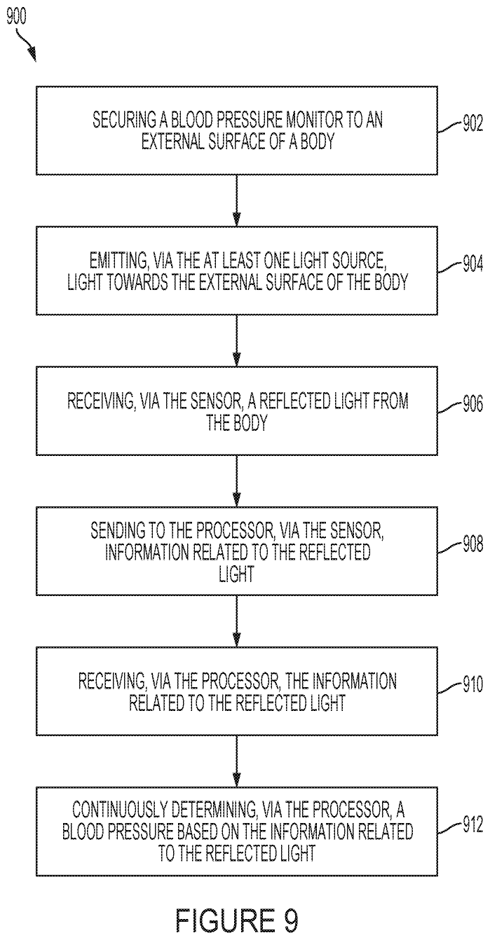

Method 900 in FIG. 9 shows an embodiment of a method that can be implemented within an operating environment including or involving, for example, exemplary blood pressure monitor system 100 of FIGS. 1-4. Method 900 includes one or more operations, functions, or actions as illustrated by one or more of blocks 902, 904, 906, 908, 910, and 912. Although the blocks are illustrated in sequential order, these blocks may also be performed in parallel, and/or in a different order than those described herein. Also, the various blocks may be combined into fewer blocks, divided into additional blocks, and/or removed based upon the desired implementation.

In addition, for the method 900 and other processes and methods disclosed herein, the flowchart shows functionality and operation of one possible implementation of some embodiments. In this regard, each block may represent a module, a segment, or a portion of program code, which includes one or more instructions executable by one or more processors for implementing specific logical functions or steps in the process. The program code may be stored on any type of computer readable medium, for example, such as a storage device including a disk or hard drive, or non-volatile memory. The computer readable medium may include non-transitory computer readable medium, for example, such as tangible, non-transitory computer-readable medium that stores data for short periods of time like register memory, processor cache and Random Access Memory (“RAM”). The computer readable medium may also include non-transitory media, such as secondary or persistent long term storage, like read only memory (“ROM”), optical or magnetic disks, compact-disc read only memory (“CD-ROM”), for example. The computer readable medium may also be any other volatile or non-volatile storage systems. The computer readable medium may be considered a computer readable storage medium, for example, or a tangible storage device. In addition, for the method 900 and other processes and methods disclosed herein, each block in FIG. 9 may represent circuitry that is wired to perform the specific logical functions in the process.

Method 900 includes securing the previously described blood pressure monitor 100 to an external surface of a body, as shown by block 902. Next, at least one light source 106 emits light towards the external surface of the body, as shown by block 904. Then, a sensor 102 receives a reflected light from the body, as shown by block 906. The sensor 102 then sends to the processor 110 information related to the reflected light, as shown by block 908. The processor 110 then receives the information related to the reflected light, as shown by block 910. And the processor 110 continuously determines a blood pressure based on the information related to the reflected light, as shown by block 912. As described above, “information related to the reflected light” includes the recorded pulse waveform cycles themselves and their associated timing. Additional, information can then be extracted from the recorded pulse waveform cycles by the processor 110 (e.g., timing of peaks and inflection points relative to the R-wave of a simultaneously recorded ECG, Volume Time Integral of the systolic and diastolic segments of the PPG waveforms and total VTI, the slopes of the rising systolic limb and the slopes of the systolic decay and the diastolic runoff) to determine blood pressure.

In one embodiment, the method 900 further includes storing one or both of the information related to the reflected light and the determined blood pressure. The information may be stored locally in the blood pressure monitor system 100 or transmitted to another device, such as a computing device, smartphone, tablet, or smart watch, for example, for remote storage or data processing.

In various embodiments, method 900 further includes the processor 110 determining whether the determined blood pressure is above a high blood pressure threshold. Then, in response to the processor 110 determining that the determined blood pressure is above the high blood pressure threshold, issuing an alert. Similarly, in some embodiments, method 900 includes the processor 110 determining whether the determined blood pressure is below a low blood pressure threshold. Then, in response to the processor 110 determining that the determined blood pressure is below the low blood pressure threshold, the processor 110 causing an alert to issue. The alert for determinations of high or low blood pressure may be one or more of (i) a visual alert, (ii) an auditory alert, or (iii) a haptic alert. In further optional embodiments, method 900 may include the processor 110 receiving patient-related data, and then, based on the received patient-related data, the processor 110 setting a high blood pressure threshold and a low blood pressure threshold. The patient related data can be supplied in a suitable manner, such as independently by a medical professional. For example, the medical professional may determine a low blood pressure threshold such as 120/80 and the high blood pressure threshold such as 140/90 for a normal patient, but for a stroke patient the high blood pressure threshold may be 133/76 or other number determined by the medical professional, for example.

In one embodiment, emitting, via the light source, the light towards the external surface of the body, includes emitting a broad-spectrum white light. The sensor then may resolve the broad-spectrum white light into a blue spectrum, a green spectrum, and a red spectrum. Next, the processor 110 measures a time difference and intensity difference between a pulse waveform cycle recorded at the red spectrum and a pulse waveform cycle recorded at the green spectrum and a pulse waveform cycle recorded at the blue spectrum. This information can then be used in the determination of blood pressure.

In another embodiment, emitting, via the light source 106, light towards an external surface of the body, includes emitting a plurality of different wavelengths of light. The at least one light source 106 includes a plurality of LEDs each corresponding to a single color of a light spectrum such that the plurality of LEDs each corresponds to a different wavelength of light. The method 900 may further include sequentially pulsing each of the plurality of LEDs. The sensor 102 then measures an intensity of the reflected light for each of the plurality of LEDs. And the processor 110 records the intensity of the reflected light for each different wavelength of light.

In some embodiments, method 900 may further include the processor 110 averaging a plurality of pulse waveform cycles to obtain an average value corresponding to each different wavelength of light. The processor 110 then calculates a first derivative, a second derivative, and a third derivative of the average value corresponding to each different wavelength of light. And the processor 110 next identifies peaks, points of inflection and aberrancy or symmetry along each of the plurality of waveform cycles.

In another embodiment, method 900 further includes a second sensor receiving a transilluminated light that has passed through the body, or alternatively a sensing device receiving transilluminated light can serve as an alternate embodiment providing the only generated data for the system. The processor uses the data associated with the transilluminated light to further determine timing of the pulse waveform cycle.

In some embodiments, the external surface of the body includes a chest, a phalanx of a finger, a forehead, an earlobe, a leg, an ankle, a toe, a wrist, or an arm. The finger and earlobe are more likely to be utilized with the transillumination embodiment because these portions of the body are thin enough for light to travel through.

In various embodiments, method 900 further includes amplifying, via a trans-impedance amplifier, a signal from the sensor. In other embodiments, method 900 further includes an analog-to-digital converter digitizing recorded signals at a sampling rate of at least 250 samples per second, for example, for each recorded pulse waveform cycle. In still further embodiments, method 900 further includes measuring an R-wave via an ECG at the same time the reflected light is received from the body by the sensor 102.

### IV. Conclusion

The description above discloses, among other things, various example systems, methods, apparatus, and articles of manufacture including, among other components, firmware and/or software executed on hardware. Such examples are merely illustrative and should not be considered as limiting. For example, it is contemplated that any or all of the firmware, hardware, and/or software aspects or components can be embodied exclusively in hardware, exclusively in software, exclusively in firmware, or in any combination of hardware, software, and/or firmware. Accordingly, the examples provided are not the only way(s) to implement such systems, methods, apparatus, and/or articles of manufacture.

As used in this application, the terms “component” and “system” are intended to refer to a computer-related entity, either hardware, a combination of hardware and software, software, or software in execution. For example, a component can be, but is not limited to being, a process running on a processor, a processor, an object, an executable, a thread of execution, a program, and/or a computer. By way of illustration, both an application running on a server and the server can be a component. One or more components can reside within a process and/or thread of execution, and a component can be localized on one computer and/or distributed between two or more computers.

Generally, program modules include routines, programs, components, data structures, etc., that perform particular tasks or implement particular abstract data types. Moreover, those skilled in the art will appreciate that the inventive methods can be practiced with other computer system configurations, including single-processor or multiprocessor computer systems, minicomputers, mainframe computers, as well as personal computers, hand-held computing devices, microprocessor-based or programmable consumer electronics, and the like, each of which can be operatively coupled to one or more associated devices.

The illustrated aspects of the innovation may also be practiced in distributed computing environments where certain tasks are performed by remote processing devices that are linked through a communications network. In a distributed computing environment, program modules can be located in both local and remote memory storage devices.

A computer typically includes a variety of computer-readable media. Computer-readable media can be any available media that can be accessed by the computer and includes both volatile and nonvolatile media, removable and non-removable media. By way of example, and not limitation, computer-readable media can comprise computer storage media and communication media. Computer storage media includes volatile and nonvolatile, removable and non-removable media implemented in any method or technology for storage of information such as computer-readable instructions, data structures, program modules or other data. Computer storage media includes, but is not limited to, RAM, ROM, EEPROM, flash memory or other memory technology, CD-ROM, digital versatile disk (DVD) or other optical disk storage, magnetic cassettes, magnetic tape, magnetic disk storage or other magnetic storage devices, or any other medium which can be used to store the desired information and which can be accessed by the computer.

Communication media typically embodies computer-readable instructions, data structures, program modules or other data in a modulated data signal such as a carrier wave or other transport mechanism, and includes any information delivery media. The term “modulated data signal” means a signal that has one or more of its characteristics set or changed in such a manner as to encode information in the signal. By way of example, and not limitation, communication media includes wired media such as a wired network or direct-wired connection, and wireless media such as acoustic, RF, infrared and other wireless media. Combinations of the any of the above should also be included within the scope of computer-readable media.

Software includes applications and algorithms. Software may be implemented in a smart phone, tablet, or personal computer, in the cloud, on a wearable device, or other computing or processing device. Software may include logs, journals, tables, games, recordings, communications, SMS messages, Web sites, charts, interactive tools, social networks, VOIP (Voice Over Internet Protocol), e-mails, and videos.

Additionally, references herein to “embodiment” means that a particular feature, structure, or characteristic described in connection with the embodiment can be included in at least one example embodiment of an invention. The appearances of this phrase in various places in the specification are not necessarily all referring to the same embodiment, nor are separate or alternative embodiments mutually exclusive of other embodiments. As such, the embodiments described herein, explicitly and implicitly understood by one skilled in the art, can be combined with other embodiments.

The specification is presented largely in terms of illustrative environments, systems, procedures, steps, logic blocks, processing, and other symbolic representations that directly or indirectly resemble the operations of data processing devices coupled to networks. These process descriptions and representations are typically used by those skilled in the art to most effectively convey the substance of their work to others skilled in the art. Numerous specific details are set forth to provide a thorough understanding of the present disclosure. However, it is understood to those skilled in the art that certain embodiments of the present disclosure can be practiced without certain, specific details. In other instances, well known methods, procedures, components, and circuitry have not been described in detail to avoid unnecessarily obscuring aspects of the embodiments. Accordingly, the scope of the present disclosure is defined by the appended claims rather than the forgoing description of embodiments.

A blood pressure monitor comprising: a housing; a sensor arranged in the housing; at least one light source arranged in the housing; and a processor in communication with the sensor, the processor configured to continuously determine a blood pressure based on a reflected light from light emitted by the at least one light source and received by the sensor. The at least one light source is an LED comprising a broad-spectrum white LED, the sensor is arranged in a first side of the housing, the at least one LED is arranged adjacent the sensor on the first side of the housing, and the housing has a barrier arranged between the sensor and the at least one LED, wherein the barrier is opaque. The at least one light source comprises a plurality of LEDs each corresponding to a single color of a light spectrum such that the plurality of LEDs each corresponds to a different wavelength of light. The sensor is a multispectral sensor. The sensor comprises a multispectral array of photodiode sensors. The different wavelength of light for each of the plurality of LEDs ranges from 425 nm to 1100 nm on the light spectrum. The blood pressure monitor further comprising: an array of trans-impedance amplifiers, wherein the array of trans-impedance amplifiers have a low noise and high common mode rejection ratio. The blood pressure monitor further comprising: at least one analog to digital converter configured to digitize recorded signals for each pulse wave of light emitted from the at least one light source at a sampling rate of at least 250 samples per second for each of the recorded pulse waves of light. The sensor and the at least one light source are spaced apart from each other a distance ranging from 1 mm to 10 mm. The housing has a central segment with three lobes extending therefrom, the sensor and the at least one light source are arranged in the central segment. The blood pressure monitor further comprising: an ECG electrode coupled to each of the three lobes configured to record at least one channel of ECG. The housing is curved to adapt to a surface of a body. The sensor and the at least one light source are arranged side by side in a first side of the housing. The sensor is arranged surrounding the at least one light source in a first side of the housing. The at least one light source is arranged surrounding the sensor in a first side of the housing. The housing is opaque. The blood pressure monitor further comprising: a band or a garment coupled to the housing and configured to retain the housing in place on a body or an adhesive disposed on the first surface of the housing configured to adhere the housing to a surface of a body. The blood pressure monitor comprising: a force sensitive resistor configured to record and register a contact pressure between the sensor and a surface of a body part. The blood pressure monitor further comprising: a second sensor arranged opposite to the at least one light source wherein a portion of a body may be disposed between the at least one light source and the second sensor. The blood pressure monitor further comprising: an accelerometer coupled to the housing and in communication with the processor.

A method for continuously monitoring blood pressure comprising: securing a blood pressure monitor according to any one of claims 1-20 to an external surface of a body; emitting, via the at least one light source, light towards the external surface of the body; receiving, via the sensor, a reflected light from the body; sending to the processor, via the sensor, information related to the reflected light; receiving, via the processor, the information related to the reflected light; and continuously determining, via the processor, a blood pressure based on the information related to the reflected light. The method further comprising: storing one or both of the information related to the reflected light and the determined blood pressure. The method further comprising: determining, via the processor, whether the determined blood pressure is above a high blood pressure threshold. The method further comprising: in response to determining that the determined blood pressure is above the high blood pressure threshold, the processor causing an alert to issue, wherein the alert is one or more of a (i) visual, (ii) auditory, or (iii) haptic alert. The method further comprising: determining, via the processor, whether the determined blood pressure is below a low blood pressure threshold. The method further comprising: in response to determining, via the processor, that the determined blood pressure is below the low blood pressure threshold, the processor causing an alert to issue, wherein the alert is one or more of (i) a visual alert, (ii) an auditory alert, or (iii) a haptic alert. The method further comprising: receiving, by the processor, patient-related data; and based on the received patient-related data, the processor setting a high blood pressure threshold and a low blood pressure threshold. The method wherein emitting, via the light source, the light towards the external surface of the body, comprises emitting a broad-spectrum white light. The sensor comprises a multispectral sensor, the method further comprising: resolving, via the sensor, the broad-spectrum white light into a blue spectrum, a green spectrum, and a red spectrum; and measuring, via the processor, a time difference and intensity difference between a pulse waveform cycle recorded at the red spectrum and a pulse waveform cycle recorded at the green spectrum, and a pulse waveform cycle recorded at the blue spectrum. The method wherein emitting, via the light source, light towards an external surface of the body, comprises emitting a plurality of different wavelengths of light. The at least one light source comprises a plurality of LEDs each corresponding to a single color of a light spectrum such that the plurality of LEDs each corresponds to a different wavelength of light, the method further comprising: sequentially pulsing each of the plurality of LEDs; measuring, via the sensor, an intensity of the reflected light for each of the plurality of LEDs; and recording, via the processor, the intensity of the reflected light for each different wavelength of light. The method further comprising: averaging, via the processor, a plurality of waveform cycles to obtain an average value corresponding to each different wavelength of light; calculating, via the processor, a first derivative, a second derivative, and a third derivative of the average value corresponding to each different wavelength of light; and identifying, via the processor, peaks, points of inflection and aberrancy or symmetry along each of the plurality of waveform cycles. The plurality of different wavelengths of light ranges from 425 nm to 1100 nm on a light spectrum. The method further comprising: receiving, via a second sensor, a transilluminated light that has passed through the body. The external surface of the body comprises a chest, a phalanx of a finger, a forehead, an earlobe, a leg, an ankle, a toe, a wrist, or an arm. The method further comprising: amplifying, via a trans-impedance amplifier, a signal from the sensor. The method further comprising: digitizing, via an analog-to-digital converter, recorded signals at a sampling rate of at least 250 samples per second for each recorded pulse waveform cycle. The method further comprising: measuring an R-wave via an ECG at the same time the reflected light is received from the body by the sensor. The blood pressure corresponds to a central blood pressure.

Tangible, non-transitory computer-readable medium having instructions encoded thereon, wherein the instructions, when executed by the processor, cause a blood pressure monitor to perform a method comprising: emitting, via the at least one light source, light towards the external surface of the body; receiving, via the sensor, a reflected light from the body; sending to the processor, via the sensor, information related to the reflected light; receiving, via the processor, the information related to the reflected light; and continuously determining, via the processor, a blood pressure based on the information related to the reflected light. The method further comprises: storing one or both of the information related to the reflected light and the determined blood pressure. The tangible, non-transitory computer-readable medium further comprising: determining whether the determined blood pressure is above a high blood pressure threshold. The tangible, non-transitory computer-readable medium further comprising: in response to determining that the determined blood pressure is above the high blood pressure threshold, sending an instruction thereby causing an alert to be issued, wherein the alert is one or more of (i) a visual alert, (ii) an auditory alert, or (iii) a haptic alert. The tangible, non-transitory computer-readable medium further comprising: determining whether the determined blood pressure is below a low blood pressure threshold. The tangible, non-transitory computer-readable medium further comprising: in response to determining that the determined blood pressure is below the low blood pressure threshold, issuing an alert, wherein the alert is one or more of a (i) visual, (ii) auditory, or (iii) haptic alert. The tangible, non-transitory computer-readable medium, further comprising: receiving, by the processor, patient-related data; and based on the received patient-related data, setting a high blood pressure threshold and a low blood pressure threshold. The tangible, non-transitory computer-readable medium wherein emitting, via the light source, the light towards the external surface of the body, comprises emitting a broad-spectrum white light. The tangible, non-transitory computer-readable medium wherein the sensor comprises a multispectral sensor, the method further comprising: resolving, via the sensor, the broad-spectrum white light into a blue spectrum, a green spectrum, and a red spectrum; and measuring, via the processor, a time difference and intensity difference between a pulse waveform recorded at the red spectrum and a pulse waveform recorded at the blue spectrum or the green spectrum. The tangible, non-transitory computer-readable medium wherein emitting, via the light source, light towards an external surface of the body, comprises emitting a plurality of different wavelengths of light. The tangible, non-transitory computer-readable medium wherein the at least one light source is a light emitting diode that comprises a plurality of LEDs each corresponding to a single color of a light spectrum such that the plurality of LEDs each corresponds to a different wavelength of light, the method further comprising: sequentially pulsing each of the plurality of LEDs; measuring, via the sensor, an intensity of the reflected light for each of the plurality of LEDs; and recording, via the processor, the intensity of the reflected light for each different wavelength of light. The tangible, non-transitory computer-readable medium further comprising: averaging a plurality of waveform cycles to obtain an average value corresponding to each different wavelength of light; calculating a first derivative, a second derivative, and a third derivative of the average value corresponding to each different wavelength of light; and identifying peaks, points of inflection and aberrancy or symmetry along each of the plurality of waveform cycles. The tangible, non-transitory computer-readable medium, further comprising: receiving, via a second sensor, a transilluminated light that has passed through the body. The tangible, non-transitory computer-readable medium wherein the external surface of the body comprises a chest, a phalanx of a finger, a forehead, an earlobe, a leg, an ankle, a toe, a wrist, or an arm. The tangible, non-transitory computer-readable medium, further comprising: amplifying, via a trans-impedance amplifier, a signal from the sensor. The tangible, non-transitory computer-readable medium further comprising: digitizing, via an analog-to-digital converter, recorded signals at a sampling rate of at least 250 samples per second for each recorded waveform cycle. The tangible, non-transitory computer-readable medium further comprising: measuring an R-wave via an ECG at the same time the reflected light is received from the body by the sensor. The tangible, non-transitory computer-readable medium wherein the blood pressure corresponds to a central blood pressure, and to stroke volume and cardiac output.

When any of the appended claims are read to cover a purely software and/or firmware implementation, at least one of the elements in at least one example is hereby expressly defined to include a tangible, non-transitory medium such as a memory, DVD, CD, Blu-ray, and so on, storing the software and/or firmware.

What is claimed is:

1. A blood pressure monitor comprising:
a housing;
a sensor arranged in the housing;
at least one light source arranged in the housing; and
a processor in communication with the sensor, the processor configured to continuously determine a blood pressure based on a reflected light from light emitted by the at least one light source and received by the sensor.

2. The blood pressure monitor of claim 1, wherein the at least one light source is an LED comprising a broad-spectrum white LED, the sensor is arranged in a first side of the housing, the at least one LED is arranged adjacent the sensor on the first side of the housing, and the housing has a barrier arranged between the sensor and the at least one LED, wherein the barrier is opaque.

3. The blood pressure monitor of claim 1, wherein the at least one light source comprises a plurality of LEDs each corresponding to a single color of a light spectrum such that the plurality of LEDs each corresponds to a different wavelength of light; and wherein the sensor is a multispectral sensor

4. The blood pressure monitor of claim 3, wherein the different wavelength of light for each of the plurality of LEDs ranges from 425 nm to 1100 nm on the light spectrum.

5. The blood pressure monitor of any one of claim 6, further comprising:
an array of trans-impedance amplifiers, wherein the array of trans-impedance amplifiers have a low noise and high common mode rejection ratio.

6. The blood pressure monitor of claim 7, further comprising:
at least one analog to digital converter configured to digitize recorded signals for each pulse wave of light emitted from the at least one light source at a sampling rate of at least 250 samples per second for each of the recorded pulse waves of light.

7. The blood pressure monitor of claim 8, wherein the sensor and the at least one light source are spaced apart from each other a distance ranging from 1 mm to 10 mm.

8. The blood pressure monitor of any one of claim 9, wherein the housing has a central segment with three lobes extending therefrom, the sensor and the at least one light source are arranged in the central segment.

9. The blood pressure monitor of claim 10, further comprising:
an ECG electrode coupled to each of the three lobes configured to record at least one channel of ECG.

10. The blood pressure monitor of claim 9, further comprising:
a band or a garment coupled to the housing and configured to retain the housing in place on a body or an adhesive disposed on the first surface of the housing configured to adhere the housing to a surface of a body.

11. The blood pressure monitor of claim 10, further comprising:
a second sensor arranged opposite to the at least one light source wherein a portion of a body may be disposed between the at least one light source and the second sensor.

12. A method for continuously monitoring blood pressure comprising:
securing a blood pressure monitor to an external surface of a body;
emitting, via the at least one light source, light towards the external surface of the body;
receiving, via the sensor, a reflected light from the body;
sending to the processor, via the sensor, information related to the reflected light;
receiving, via the processor, the information related to the reflected light
recording pulsewave parameters and characteristics at different depths of the skin; and
continuously determining, via the processor, a blood pressure based on the acquisition and analysis of different pulse-waveforms corresponding to different wavelengths related to the reflected light.

13. The method of claim 21, further comprising:
storing one or both of the information related to the reflected light and the determined blood pressure; and recording the time differences between different waveforms at different depths to correspond to the pulse wave propagation time from deeper arteries and arterioles up to surface capillaries; recording the differences in the pulsewave intensities including as flux, quantity, magnitude, or volume, at different depths and recording the resistance to flow at different levels of the arteries and the variability of the pulsewave intensities over time.

14. The method of one of claim 21 or 22, further comprising:
determining, via the processor, whether the determined blood pressure is above a high blood pressure threshold or below a low blood pressure threshold; the at least one light source is a white broadspectrum light source; and resolving the reflected light into different spectra using a sensor that resolves the spectrum of light based on spectrophotometry principles or based on photodiodes covered by a plurality of spectral filters.

15. The method of claim 14, wherein the sensor comprises a multispectral sensor, the method further comprising:
resolving, via the sensor, the broad-spectrum white light into a blue spectrum, a green spectrum, and a red spectrum; and
measuring, via the processor, a time difference and intensity difference between a pulse waveform cycle recorded at the red spectrum and a pulse waveform cycle recorded at the green spectrum, and a pulse waveform cycle recorded at the blue spectrum.

16. The method of claim 15, wherein emitting, via the light source, light towards an external surface of the body, comprises emitting a plurality of different wavelengths of light.

17. The method of claim 16, wherein the at least one light source comprises a plurality of LEDs each corresponding to a single color of a light spectrum such that the plurality of LEDs each corresponds to a different wavelength of light, the method further comprising:
sequentially pulsing each of the plurality of LEDs;
measuring, via the sensor, an intensity of the reflected light for each of the plurality of LEDs; and
recording, via the processor, the intensity of the reflected light for each different wavelength of light.

18. The method of claim 17, further comprising:
averaging, via the processor, a plurality of waveform cycles to obtain an average value corresponding to each different wavelength of light;
calculating, via the processor, a first derivative, a second derivative, and a third derivative of the average value corresponding to each different wavelength of light; and
identifying, via the processor, peaks, points of inflection and aberrancy or symmetry along each of the plurality of waveform cycles.

19. The method of claim 18, further comprising:
digitizing, via an analog-to-digital converter, recorded signals at a sampling rate of at least 250 samples per second for each recorded pulse waveform cycle.

20. Tangible, non-transitory computer-readable medium having instructions encoded thereon, wherein the instructions, when executed by the processor, cause a blood pressure monitor to perform a method comprising:
emitting, via the at least one broad-spectrum white light source including a plurality of different wavelengths of light, light towards the external surface of the body;
receiving, via the sensor, a reflected light from the body;
sending to the processor, via the sensor, information related to the reflected light;
receiving, via the processor, the information related to the reflected light;
continuously determining, via the processor, a blood pressure based on the information related to the reflected light; storing one or both of the information related to the reflected light and the determined blood pressure;
the sensor comprises a multispectral sensor, the method further comprising:
resolving, via the sensor, the broad-spectrum white light into a blue spectrum, a green spectrum, and a red spectrum; and
measuring, via the processor, a time difference and intensity difference between a pulse waveform recorded at the red spectrum and a pulse waveform recorded at the blue spectrum or the green spectrum;
measuring, via the sensor, an intensity of the reflected light for each of the plurality of LEDs;
recording, via the processor, the intensity of the reflected light for each different wavelength of light;
averaging a plurality of waveform cycles to obtain an average value corresponding to each different wavelength of light;
calculating a first derivative, a second derivative, and a third derivative of the average value corresponding to each different wavelength of light; and
identifying peaks, points of inflection and aberrancy or symmetry along each of the plurality of waveform cycles;
digitizing, via an analog-to-digital converter, recorded signals at a sampling rate of at least 250 samples per second for each recorded waveform cycle.
